(19) United States
(12) Patent Application Publication
(10) Pub. No.: US 2024/0108411 A1
(43) Pub. Date: Apr. 4, 2024

(54) SUCTION SYSTEM AND ENDOSCOPE

(71) Applicant: OLYMPUS MEDICAL SYSTEMS CORP., Tokyo (JP)

(72) Inventor: Takumi KAWAKAMI, Tokyo (JP)

(73) Assignee: OLYMPUS MEDICAL SYSTEMS CORP., Tokyo (JP)

(21) Appl. No.: 17/957,120

(22) Filed: Sep. 30, 2022

(51) Int. Cl. A61B 18/26

(52) U.S. Cl. CPC A61B 18/26; A61B 2018/206; A61B 2218/007

(57) ABSTRACT

A suction system of an endoscope includes a branch conduit including a suction pipe sleeve branching from a main conduit; a suction path provided in the suction pipe sleeve and including a first opening communicating with the main conduit; a working channel including an insertion path communicating with the main conduit; and a liquid feed tube inserted through the main conduit and the working channel. A first minimum length A, a maximum length B, and a second minimum length C have a dimensional relation of C−B<(A−B)/2, the first minimum length A passing through a first center on a plane of the first opening, the maximum length B passing through a second center on a cross section orthogonal to a longitudinal direction of the liquid feed tube, the second minimum length C passing through a third center on a cross section orthogonal to a longitudinal direction of the insertion path.

### BACKGROUND OF THE INVENTION

### 1. Field of the Invention

The present invention relates to a suction system used in combination with an endoscope, and an endoscope.

### 2. Description of the Related Art

A medical device is well-known that is used in combination with an endoscope to be inserted into a living organism and is adapted to collect a solid object present in the living organism together with a fluid.

For example, Japanese Patent Application Laid-Open Publication No. 2016-67702 discloses a technique of a medical device including a cylindrical body having a suction port and a discharge port, an impeller configured to carry a fluid from the suction port to the discharge port, and a filter configured to collect a solid object.

Meanwhile, for example, Japanese Patent Application Laid-Open Publication No. 2014-23945 discloses a technique of an endoscope apparatus including an endoscope having a treatment instrument channel that allows for the passage of a perfusion/suction channel and a stone retrieval basket, and also including branch conduits.

### SUMMARY OF THE INVENTION

A suction unit according to one embodiment of the present invention includes a branch conduit including a main conduit and a suction pipe sleeve branching from the main conduit; a suction path provided in the suction pipe sleeve, the suction path including a first opening communicating with the main conduit; a working channel including an insertion path communicating with the main conduit; and a liquid feed tube inserted through the main conduit and the working channel, in which a first minimum length A, a maximum length B, and a second minimum length C have a dimensional relation of C−B<(A−B)/2, the first minimum length A passing through a first center on a plane of the first opening, the maximum length B passing through a second center on a cross section orthogonal to a longitudinal direction of the liquid feed tube, the second minimum length C passing through a third center on a cross section orthogonal to a longitudinal direction of the insertion path.

An endoscope according to one embodiment of the present invention includes an insertion portion inserted into a body cavity; an operation portion provided continuously with the insertion portion; a branch conduit provided on the operation portion, the branch conduit including a main conduit provided in a longitudinal direction of the branch conduit, and a suction pipe sleeve branching from the main conduit; a suction path provided in the suction pipe sleeve, the suction path including a first opening communicating with the main conduit; a working channel provided in the insertion portion and the operation portion, the working channel including an insertion path communicating with the main conduit; and a liquid feed tube inserted through the main conduit and the working channel, in which a first minimum length A, a maximum length B, and a second minimum length C have a dimensional relation of C−B<(A−B)/2, the first minimum length A passing through a first center on a plane of the first opening, the maximum length B passing through a second center on a cross section orthogonal to a longitudinal direction of the liquid feed tube, the second minimum length C passing through a third center on a cross section orthogonal to a longitudinal direction of the insertion path.

### BRIEF DESCRIPTION OF THE DRAWINGS

FIG. 1 is a perspective view illustrating a configuration of an endoscope including a suction system;

FIG. 2 is a view illustrating a schematic configuration of the suction system;

FIG. 3 is a cross-sectional view illustrating a configuration of a branch conduit to which a working channel is connected;

FIG. 4 is a perspective view illustrating a distal end portion of an insertion portion from which a liquid feed tube extends;

FIG. 5 is a cross-sectional view along line V-V in FIG. 3 of a suction pipe sleeve of the branch conduit through which the liquid feed tube is inserted;

FIG. 6 is a cross-sectional view along line VI-VI in FIG. 3 of the working channel through which the liquid feed tube is inserted;

FIG. 7 is a perspective view illustrating a distal end portion of an insertion portion from which a liquid feed tube extends according to a first modification;

FIG. 8 is a cross-sectional view of a suction pipe sleeve of a branch conduit through which a liquid feed tube is inserted according to a second modification;

FIG. 9 is a cross-sectional view of a suction pipe sleeve of a branch conduit through which a liquid feed tube is inserted according to a third modification;

FIG. 10 is a partial cross-sectional view of a branch conduit provided with a spacer according to a fourth modification;

FIG. 11 is a perspective view illustrating a liquid feed tube and a rail according to a fifth modification;

FIG. 12 is a partial cross-sectional view of a branch conduit provided with a ring member according to a sixth modification;

FIG. 13 is a perspective view illustrating a liquid feed tube and a cylindrical body according to a seventh modification; and

FIG. 14 is a partial cross-sectional view of a branch conduit illustrating a magnetic body provided on a liquid feed tube and a magnet according to an eighth modification.

### DETAILED DESCRIPTION OF THE PREFERRED EMBODIMENT

Hereinafter, a configuration of one embodiment of the present invention will be described. It should be noted that in the following description, a drawing based on each embodiment is only schematic, and thus, the relationship between the thickness and width of each portion, the proportion of the thickness of each portion, and the like are different from the actual ones. There may also be a case where portions included in different drawings have different dimensional relations, proportions, and the like.

Among ureteral stones, the largest one measures about 20 mm in diameter. Urethral lithotripsy is known that includes crushing ureteral stones into particles with a diameter of less than about 1 mm using a laser, and sucking the particles through an endoscope so as to collect the particles to the outside of the body. In such ureteral lithotripsy, it has been conventionally desired that particles resulting from the crushing of ureteral stones be smoothly collected to the outside of the body using an endoscope.

Accordingly, it is an object of the present invention to provide a suction system that can smoothly collect particles to be collected, such as crushed stones, to the outside of the body using an endoscope.

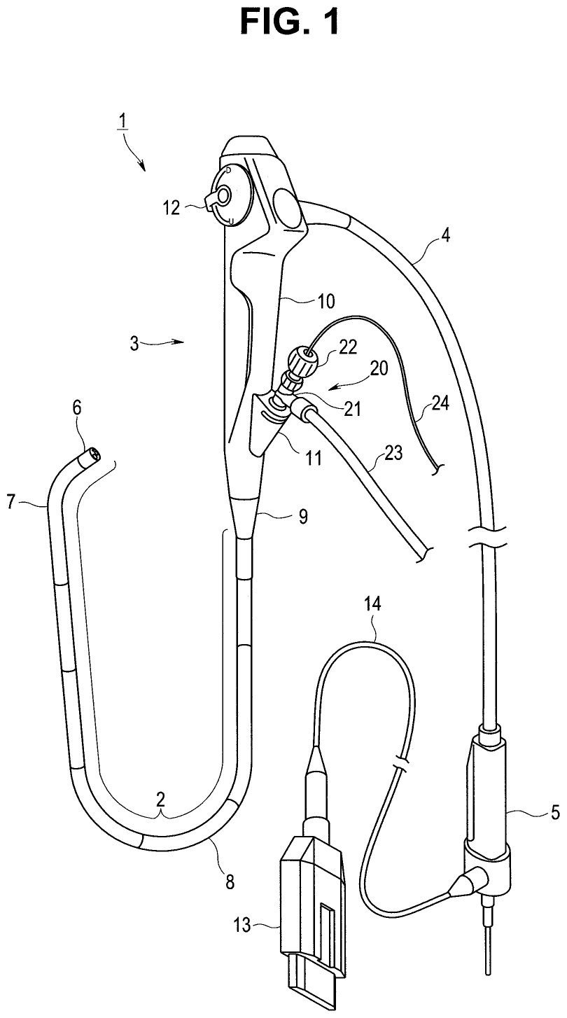

Hereinafter, an endoscope 1 of the present embodiment will be described. Note that a ureteropelvic videoscope is illustrated as an example of the endoscope 1 herein. The endoscope 1 illustrated in FIG. 1 includes an insertion portion 2, an operation portion 3, a universal cord 4, and an endoscope connector 5.

The insertion portion 2 is formed in an elongated tubular shape. The operation portion 3 is provided continuously with a proximal end of the insertion portion 2. The universal cord 4 is an endoscope cable extending from the operation portion 3. The endoscope connector 5 is provided at an end portion of the universal cord 4.

The insertion portion 2 includes a distal end portion 6, a bending portion 7, and a flexible tube portion 8 that are provided in a continuous manner in this order from the distal end side. The insertion portion 2 is a flexible tubular member.

An image pickup unit (not illustrated) incorporating an image sensor, such as a CCD or a CMOS, is provided in the distal end portion 6. The distal end portion 6 also incorporates an illumination optical system, for example.

The bending portion 7 is a bending mechanism configured to bend actively. The bending portion 7 is allowed to bend in two directions including upward and downward directions (UP-DOWN) through an operation of rotating a bending lever 12 of the operation portion 3.

Note that the bending portion 7 may also be configured to bend in four directions including leftward and rightward directions in addition to the upward and downward directions (i.e., the entire circumferential direction around the axis: UP-DOWN/RIGHT-LEFT through operations of bending the bending portion 7 in upward, downward, leftward, and rightward directions).

The flexible tube portion 8 flexes passively. The flexible tube portion 8 is a flexible tubular member.

A working channel 15, which has functions of a treatment instrument insertion channel and a suction channel, is provided in the insertion portion 2. Further, an image pickup cable, a light guide, and the like (which are not illustrated) incorporated in the distal end portion 6 are also provided in the insertion portion 2.

The operation portion 3 includes a bend preventing portion 9 connected to the flexible tube portion 8. The operation portion 3 includes a grasping portion 10 provided continuously with the bend preventing portion 9. An outer surface of the grasping portion 10 is provided with a treatment instrument insertion portion 11 as an opening for endoscope forceps. The treatment instrument insertion portion 11 is provided with a branch unit 20. Note that the detailed configuration of the branch unit 20 will be described later.

The universal cord 4 is a composite cable through which an image pickup cable, a light guide, and the like are inserted and disposed. The endoscope connector 5 is connected to a light source device (not illustrated) of an external apparatus. A signal cable 14 is connected to the endoscope connector 5. An end portion of the signal cable 14 is provided with an electrical connector 13. The electrical connector 13 is connected to a video processor (not illustrated) of an external apparatus.

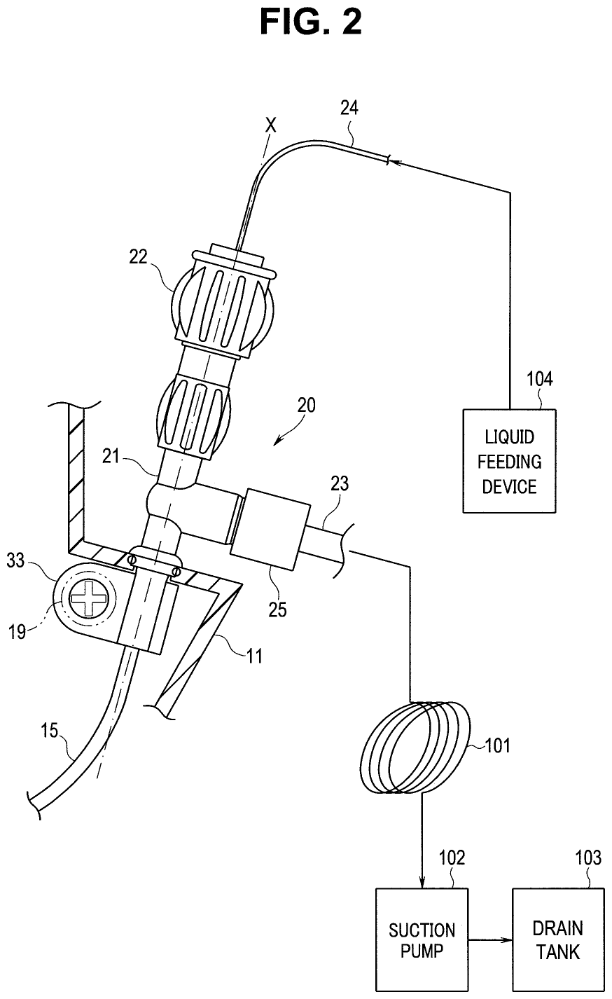

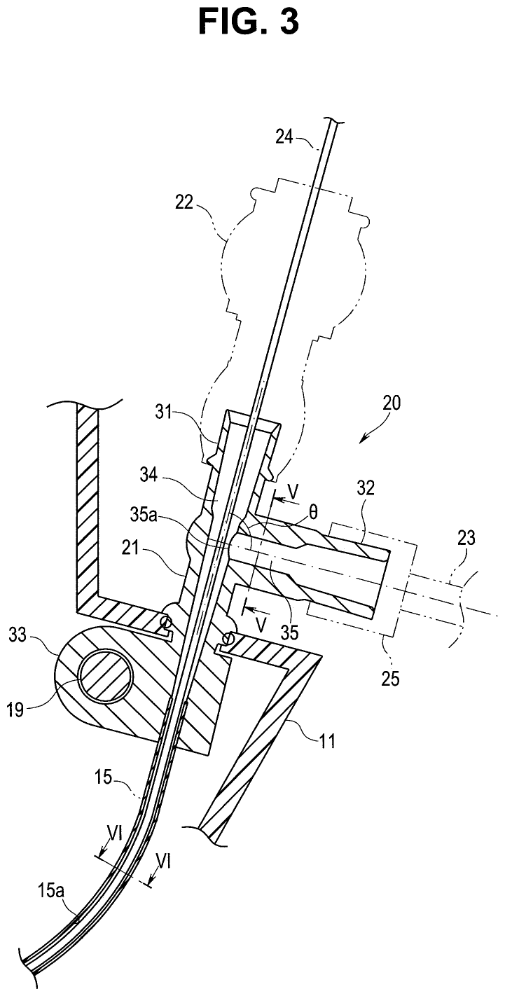

Next, a configuration of a suction system of the endoscope 1 of the present embodiment will be described below. The branch unit 20 includes a branch conduit 21 and a biopsy port seal 22 as illustrated in FIGS. 2 and 3.

The branch conduit 21 is a substantially T-shaped pipe sleeve including a liquid feed pipe sleeve 31 and a suction pipe sleeve 32. The branch conduit 21 includes a main conduit 34 and a sub-conduit 35 of a suction path. The main conduit 34 forms a treatment instrument insertion conduit and a liquid feed conduit along the longitudinal axis X of the branch conduit 21. The main conduit 34 herein is in the shape of a spindle with a hole diameter increasing toward the proximal end side.

The sub-conduit 35 is formed in the suction pipe sleeve 32. An angle θ at which the central axis of the main conduit 34 and the central axis of the sub-conduit 35 cross is greater than or equal to 60 degrees and less than or equal to 120 degrees. The sub-conduit 35 includes an opening 35a (i.e., a first opening) communicating with the main conduit 34.

The branch conduit 21 includes the liquid feed pipe sleeve 31, which also serves as a treatment instrument insertion pipe sleeve, at a proximal end along the longitudinal axis X. The main conduit 34 is provided in the liquid feed pipe sleeve 31. The liquid feed pipe sleeve 31 includes an opening of the main conduit 34. The biopsy port seal 22 is connected to the liquid feed pipe sleeve 31.

The branch conduit 21 includes the suction pipe sleeve 32 substantially orthogonal to the longitudinal axis X. The sub-conduit 35 is provided in the suction pipe sleeve 32. The suction pipe sleeve 32 includes an opening of the sub-conduit 35. A connector 25 of a suction tube 23 is connected to the suction pipe sleeve 32.

A fixation portion 33 provided on the distal end side of the branch conduit 21 is fixed in the treatment instrument insertion portion 11 by a fixation member 19, such as a screw. The branch conduit 21 is provided in the treatment instrument insertion portion 11 in a watertight (i.e., airtight) manner using a seal, such as an O-shaped ring.

Note that the working channel 15 as the treatment instrument insertion channel is connected to the distal end of the branch conduit 21. The working channel 15 communicates with the main conduit 34. The working channel 15 is provided in the insertion portion 2 through the operation portion 3.

A strainer 101 is provided in an intermediate portion of the suction tube 23. Particles of stones to be collected, which have been crushed with a laser and sucked through the suction tube 23, accumulate (i.e., pile up) in the strainer 101. Note that the particles to be collected that have accumulated in the strainer 101 are discarded or collected as a specimen after the completion of the procedures with the endoscope 1.

The suction tube 23 is connected to a suction pump 102. The suction pump 102 is connected to a drain tank 103.

The biopsy port seal 22 is removably attached to the liquid feed pipe sleeve 31 of the branch conduit 21. The biopsy port seal 22 is an accessory for endoscope that prevents back-flow of a liquid and the like by turning a nut and closing an inner seal.

Herein, a liquid feed tube 24 is inserted through a port at a proximal end of the biopsy port seal 22. For the biopsy port seal 22, the nut is turned so that the inner seal is tightly attached to an outer periphery of the liquid feed tube 24.

Accordingly, the liquid feed tube 24 is held in the biopsy port seal 22. In such a state, the biopsy port seal 22 prevents back-flow of a liquid and the like from the branch conduit 21. Note that since the biopsy port seal 22 is well-known, the description of the detailed configuration of the biopsy port seal 22 is omitted.

A proximal end side of the liquid feed tube 24 is connected to a liquid feeding device 104 of an external apparatus. The liquid feeding device 104 sends a biologically compatible solution, such as physiological saline, into the liquid feed tube 24.

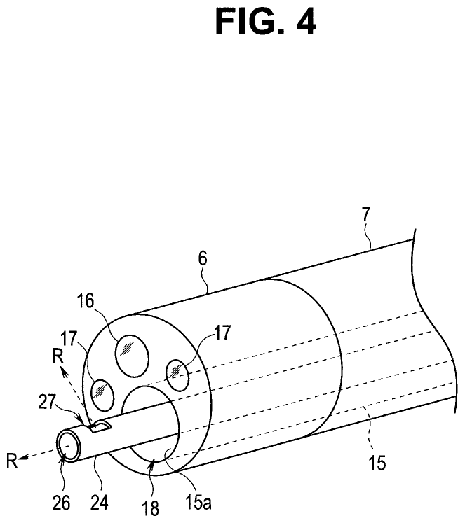

The liquid feed tube 24 is inserted into the branch conduit 21 through the biopsy port seal 22. The liquid feed tube 24 is inserted through the working channel 15, and is inserted up to the distal end portion 6 of the insertion portion 2. As illustrated in FIG. 4, the liquid feed tube 24 is led into a body of a subject through a channel opening 18 provided in the distal end portion 6.

The liquid feed tube 24 includes a distal end opening 26 and a lateral opening 27 provided near a distal end of the liquid feed tube 24. A liquid R, such as physiological saline, sent through the liquid feed tube 24 is ejected into the body from the distal end opening 26 and the lateral opening 27. As the liquid R is ejected from the lateral opening 27, convection that stirs a region in a body cavity can be generated. Note that a distal end face of the distal end portion 6 of the insertion portion 2 is provided with an observation window 16 and two illumination windows 17.

The suction system of the endoscope 1 with the foregoing configuration includes at least the working channel 15, the branch conduit 21, the suction tube 23, and the liquid feed tube 24.

Hereinafter, the size (i.e., dimensional) relation between the working channel 15 and the branch conduit 21 will be described.

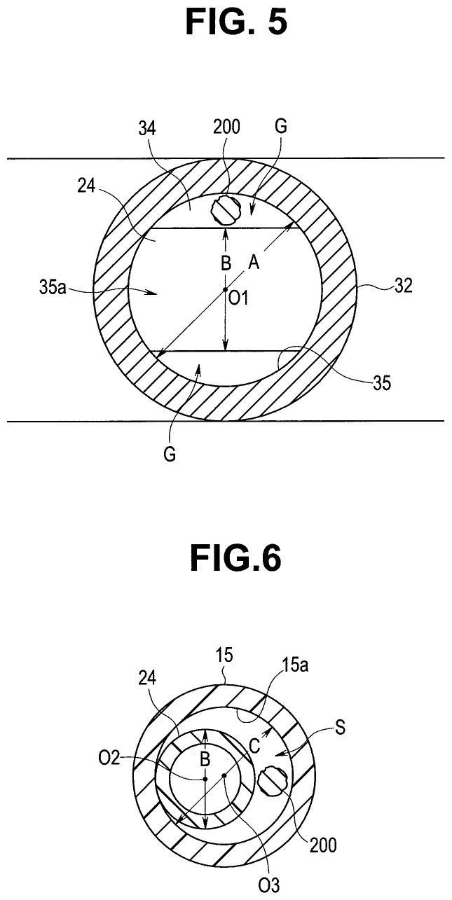

In the branch conduit 21, as illustrated in FIG. 5, the opening 35a of the sub-conduit 35 has a diameter A with the minimum length passing through a center O1 on a plane of the opening 35a. Note that since the cross section of the main conduit 34 is circular, the plane of the opening 35a is curved along an arc of the main conduit 34.

As illustrated in FIG. 5 and FIG. 6, the liquid feed tube 24 has a diameter B in a cross-sectional direction orthogonal to the longitudinal direction. An insertion path 15a in the working channel 15 has a diameter C.

Note that since the shape of the opening 35a is circular herein, the diameter A has the minimum length passing through the center O1. The diameter A of the opening 35a is the minimum inside diameter in a cross section orthogonal to the longitudinal direction of the suction pipe sleeve 32.

In other words, the suction pipe sleeve 32 has the minimum inside diameter A in a cross-sectional direction orthogonal to the longitudinal direction. In addition, the sub-conduit 35 has the minimum diameter A in a cross-sectional direction orthogonal to the longitudinal direction.

Since the outer shape of the liquid feed tube 24 is also circular herein, the diameter B has the maximum length passing through a center O2. In other words, the liquid feed tube 24 has the maximum outside diameter B in the cross-sectional direction orthogonal to the longitudinal direction.

Since the outer shape of the insertion path 15a is also circular herein, the diameter C has the minimum length passing through a center O3. In other words, the working channel 15 has the minimum inside diameter C in the cross-sectional direction orthogonal to the longitudinal direction. In addition, the insertion path 15a has the minimum outside diameter C in the cross-sectional direction orthogonal to the longitudinal direction.

In a state where the liquid feed tube 24 is inserted through the insertion path 15a of the working channel 15, a gap S between the insertion path 15a and the liquid feed tube 24 becomes maximum when the liquid feed tube 24 becomes eccentric with respect to the insertion path 15a. Therefore, a maximum gap S between the insertion path 15a and the liquid feed tube 24 is a difference (C−B) between the diameter C of the insertion path 15a and the diameter B of the liquid feed tube 24.

Thus, considering a case where the liquid feed tube 24 becomes eccentric in the working channel 15 when particles 200 to be collected, such as particles of stones crushed with a laser, are sucked from a body of a subject through the working channel 15 together with a liquid sent through the liquid feed tube 24, the maximum size of the particles 200 to be collected that can be sucked through the insertion path 15a is less than or equal to the foregoing difference (C−B).

The branch conduit 21 needs to be designed such that the particles 200 to be collected with the maximum size (C−B) can pass through the opening 35a of the sub-conduit 35 communicating with the main conduit 34.

When suction is performed while the liquid feed tube 24 is inserted through the branch conduit 21, the liquid feed tube 24 is drawn to the side of the opening 35a of the sub-conduit 35. Therefore, the opening 35a is partially blocked by the liquid feed tube 24.

In other words, when the liquid feed tube 24 is drawn to the side of the opening 35a, one or two gaps G are produced in the opening 35a depending on the position of the liquid feed tube 24. Note that when the liquid feed tube 24 is drawn to only one side, a single large gap G or two gaps G with different sizes (i.e., a large gap G and a small gap G) is/are produced in the opening 35a.

In order for the particles 200 to be collected with the maximum size to enter the sub-conduit 35 through the opening 35a, the gap(s) G in the opening 35a to which the liquid feed tube 24 is drawn need(s) to be larger than the particles 200 to be collected.

Regarding the gap(s) G in the opening 35a, two gaps G with substantially the same shape are produced when the liquid feed tube 24 is drawn so as to cover the center of the opening 35a. At this time, it is acceptable as long as the particles 200 to be collected with the maximum size can pass through the opening 35a.

Note that the single large gap G or the larger one of the two gaps G is larger than each of the two gaps G with substantially the same shape. Therefore, it is acceptable as long as the particles 200 to be collected with the maximum size can pass through one of the two gaps G produced when the liquid feed tube 24 is drawn so as to cover the center of the opening 35a. In other words, as long as the particles 200 to be collected with the maximum size can pass through the opening 35a via one of the two gaps G, the particles 200 to be collected can be sucked into the sub-conduit 35.

When the liquid feed tube 24 is drawn so as to cover the center of the opening 35a, one of the two gaps G in the opening 35a, which is not covered with the liquid feed tube 24, has half the difference (A−B) between the diameter A of the opening 35a and the diameter B of the liquid feed tube 24 [(A−B)/2].

Therefore, if the half the difference (A−B) between the diameter A of the opening 35a and the diameter B of the liquid feed tube 24 [(A−B)/2] is set larger than the maximum size (C−B) of the particles 200 to be collected, it is possible to allow the particles 200 to be collected with the maximum size to be sucked into the sub-conduit 35.

Thus, the size (i.e., dimensional) relation between the working channel 15 and the branch conduit 21 is set so that the difference (C−B) between the diameter C of the insertion path 15a and the diameter B of the liquid feed tube 24 is smaller than the half {(A−B)/2} the difference (A−B) between the diameter A of the opening 35a and the diameter B of the liquid feed tube 24 {C−B<(A−B)/2}.

Accordingly, with the suction system of the endoscope 1, even in a state where the liquid feed tube 24 is drawn to the side of the opening 35a of the sub-conduit 35 due to suction and thus becomes eccentric to one side in the branch conduit 21, the particles 200 to be collected, such as crushed particles, can be sucked without clogging the branch conduit 21.

In other words, setting the size (i.e., dimensional) relation between the working channel 15 and the branch conduit 21 to {C−B<(A−B)/2} as described above can, even when the liquid feed tube 24 is drawn to the side of the opening 35a of the sub-conduit 35 on the suction side in the branch conduit 21 and thus partially blocks the suction path, prevent a decrease in the suction efficiency without the particles 200 to be collected, such as crushed particles, clogging the branch conduit 21.

Further, with the suction system of the endoscope 1, it is possible to allow the particles 200 to be collected, such as crushed particles, to be sucked and collected through perfusion in which liquid feeding and suction are performed at the same time in a state where the liquid feed tube 24 is inserted through the branch conduit 21. Accordingly, the endoscope 1 has a configuration including a suction system that can smoothly collect the particles 200 to be collected, such as crushed stones, to the outside of the body.

### (Modifications)

### (First Modification)

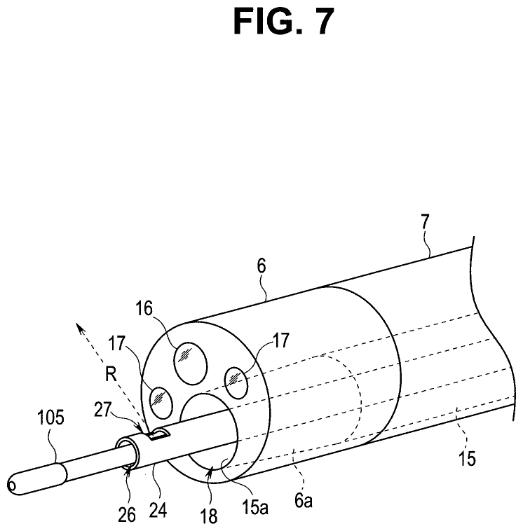

As illustrated in FIG. 7, the distal end portion 6 includes a conduit 6a communicating with the channel opening 18. The working channel 15 is bonded to the conduit 6a. The conduit 6a is formed such that an inner peripheral face of the conduit 6a is tapered toward the channel opening 18.

The channel opening 18 has the diameter C set in the cross-sectional direction orthogonal to the longitudinal direction as described above. Note that the diameter C is the diameter of the smallest cross section among cross sections orthogonal to the longitudinal direction of the conduit 6a. Setting the diameter of the working channel 15 to be larger than the diameter C of the conduit 6a can prevent clogging of the working channel 15 with the particles 200 to be collected, such as crushed particles.

Reducing the diameter of the channel opening 18 can reduce the diameter of the distal end portion 6 of the insertion portion 2.

Further, herein, an embodiment is exemplarily illustrated in which a laser probe 105 is inserted through the liquid feed tube 24. The endoscope 1 can perform liquid feeding and suction while stones are crushed with the laser probe 105. At this time, the liquid feed tube 24 ejects the liquid R, such as physiological saline, from the lateral opening 27. Accordingly, convection is produced in a body cavity by the liquid R, and thus, the particles 200 to be collected, such as crushed particles, can be efficiently sucked into the working channel 15.

### (Second Modification)

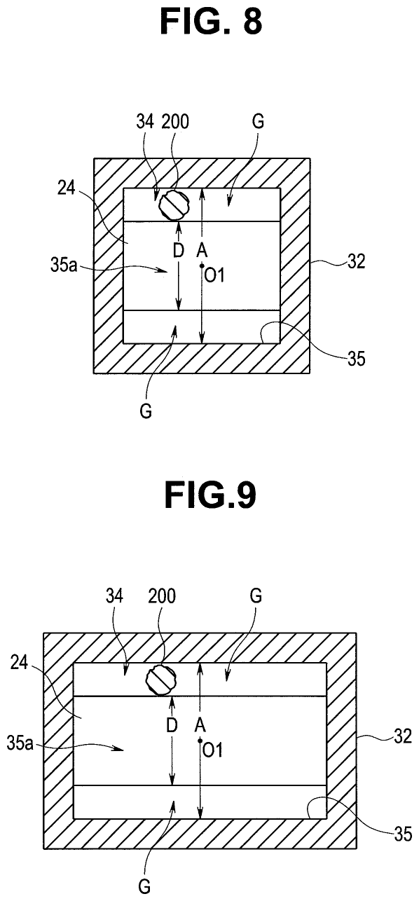

As illustrated in FIG. 8, the branch conduit 21 may include the suction pipe sleeve 32 with a square cross section. The sub-conduit 35 communicating with the main conduit 34 and the opening 35a of the suction pipe sleeve 32 are also square. Note that the length of one side of the opening 35a of the sub-conduit 35 is set equal to the foregoing diameter A.

### (Third Modification)

As illustrated in FIG. 9, the branch conduit 21 may include the suction pipe sleeve 32 with a rectangular cross section. The sub-conduit 35 communicating with the main conduit 34 and the opening 35a of the suction pipe sleeve 32 are also rectangular. Note that the length in the short-side direction of the opening 35a of the sub-conduit 35 is set equal to the foregoing diameter A.

### (Fourth Modification)

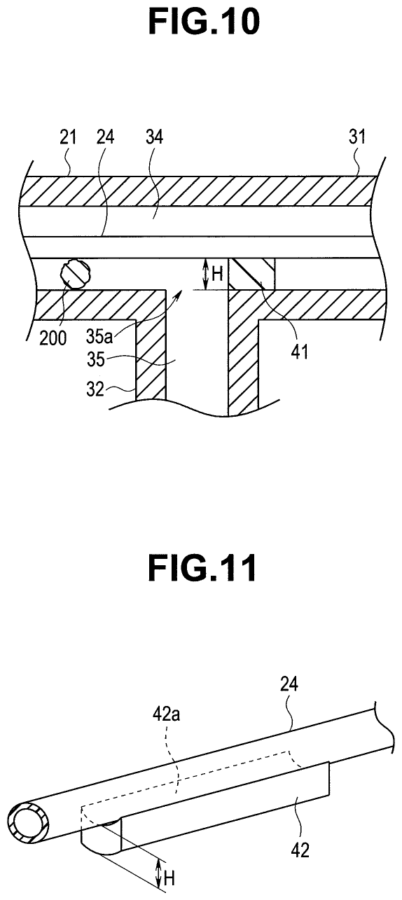

As illustrated in FIG. 10, in the branch conduit 21, a region of the main conduit 34 near the opening 35a of the sub-conduit 35, on the proximal end side with respect to the opening 35a, may be provided with a rib 41 as a position restriction member configured to restrict the position of the liquid feed tube 24. The rib 41 is a block having a predetermined height H and disposed to protrude in the central direction of the main conduit 34.

The liquid feed tube 24 touches the rib 41 and thus has a restricted position so that the liquid feed tube 24 is positioned away from the opening 35a by a predetermined distance even when drawn to the side of the opening 35a during suction. The height H of the rib 41 is set greater than the maximum size (C−B) of the particles 200 to be collected, such as crushed stones, (H>C−B).

Accordingly, the liquid feed tube 24 does not block the opening 35a, and the particles 200 to be collected easily pass through the opening 35a Thus, the particles 200 to be collected can be smoothly sucked without clogging the branch conduit 21.

### (Fifth Modification)

As illustrated in FIG. 11, in the branch conduit 21, a region of the main conduit 34 from a position near the opening 35a of the sub-conduit 35, on the proximal end side with respect to the opening 35a, to an end portion of the liquid feed pipe sleeve 31 may be provided with a rail 42 as a position restriction member. The rail 42 has a predetermined height H and is disposed to protrude in the central direction of the main conduit 34 as with the foregoing rib 41.

The rail 42 has a recessed curved face 42a, which is adapted to guide the insertion of the liquid feed tube 24, formed as a face on the protruding side.

The liquid feed tube 24 is guided by the rail 42 and thus has a restricted position so that the liquid feed tube 24 is positioned away from the opening 35a by a predetermined distance even when drawn to the side of the opening 35a during suction. The height H of the rail 42 is set greater than the maximum size (C−B) of the particles 200 to be collected, such as crushed stones, (H>C−B) as with the foregoing rib 41.

Accordingly, the liquid feed tube 24 does not block the opening 35a, and the particles 200 to be collected easily pass through the opening 35a. Thus, the particles 200 to be collected can be smoothly sucked without clogging the branch conduit 21.

### (Sixth Modification)

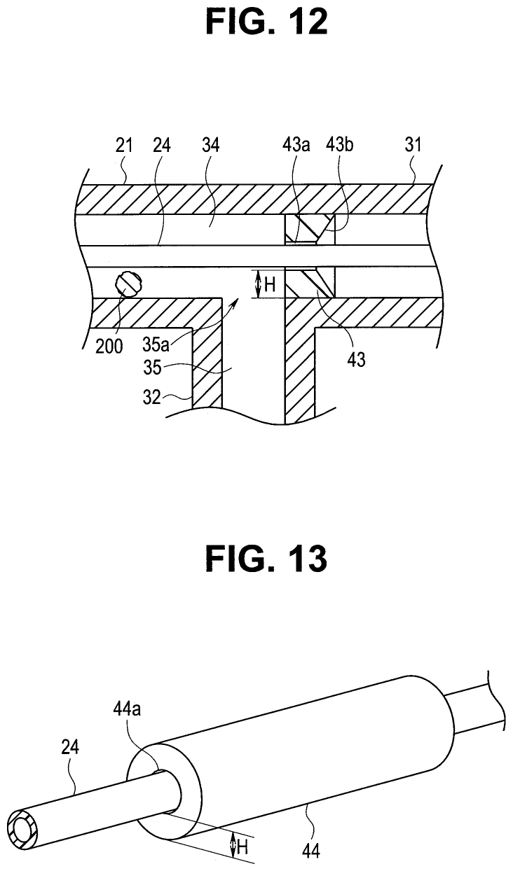

As illustrated in FIG. 12, in the branch conduit 21, a region of the main conduit 34 near the opening 35a of the sub-conduit 35, on the proximal end side with respect to the opening 35a, may be provided with a ring member 43 as a position restriction member configured to restrict the position of the liquid feed tube 24. The ring member 43 includes a hole portion 43a that allows insertion of the liquid feed tube 24.

A region from the outer periphery to the hole portion 43a of the ring member 43 has a predetermined thickness H as with the foregoing rib 41. A proximal end face of the ring member 43 is a tapered face 43b that facilitates insertion of the liquid feed tube 24 through the hole portion 43a.

The liquid feed tube 24 is inserted through the ring member 43 and thus has a restricted position so that the liquid feed tube 24 is positioned away from the opening 35a by a predetermined distance even when drawn to the side of the opening 35a during suction. The thickness H of the ring member 43 is set greater than the maximum size (C−B) of the particles 200 to be collected, such as crushed stones, (H>C−B) as with the foregoing rib 41.

Accordingly, the liquid feed tube 24 does not block the opening 35a, and the particles 200 to be collected easily pass through the opening 35a. Thus, the particles 200 to be collected can be smoothly sucked without clogging the branch conduit 21.

### (Seventh Modification)

As illustrated in FIG. 13, in the branch conduit 21, a region of the main conduit 34 from a position near the opening 35a of the sub-conduit 35, on the proximal end side with respect to the opening 35a, to an end portion of the liquid feed pipe sleeve 31 may be provided with a tubular member 44 as a position restriction member. A region from the outer periphery to a hole portion 44a of the tubular member 44 has a predetermined thickness H as with the foregoing ring member 43.

The liquid feed tube 24 is inserted through the tubular member 44 and thus has a restricted position so that the liquid feed tube 24 is positioned away from the opening 35a by a predetermined distance even when drawn to the side of the opening 35a during suction. The thickness H of the tubular member 44 is set greater than the maximum size (C−B) of the particles 200 to be collected, such as crushed stones, (H>C−B) as with the foregoing ring member 43.

Accordingly, the liquid feed tube 24 does not block the opening 35a, and the particles 200 to be collected easily pass through the opening 35a. Thus, the particles 200 to be collected can be smoothly sucked without clogging the branch conduit 21.

### (Eighth Modification)

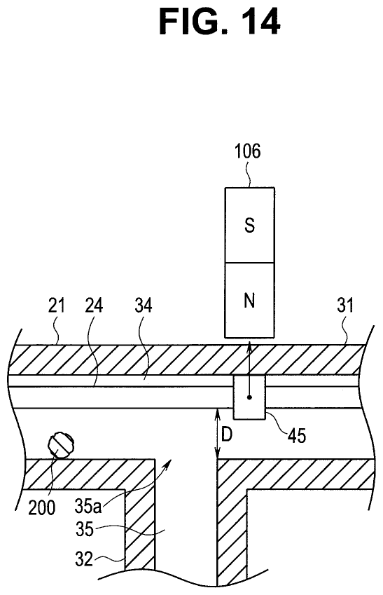

As illustrated in FIG. 14, the liquid feed tube 24 may be provided with a magnetic body 45 as a position restriction member so that the magnetic body 45 may be drawn to a side opposite to the opening 35a by a magnet 106 from outside of the branch conduit 21. Thus, even when the liquid feed tube 24 is drawn to the side of the opening 35a during suction, the liquid feed tube 24 is positioned away from the opening 35a by a predetermined distance as the magnetic body 45 is drawn to the side opposite to the opening 35a.

Note that a distance D between the liquid feed tube 24 and the opening 35a is set greater than the maximum size (C−B) of the particles 200 to be collected, such as crushed stones, (D>C−B).

Accordingly, the liquid feed tube 24 does not block the opening 35a, and the particles 200 to be collected easily pass through the opening 35a. Thus, the particles 200 to be collected can be smoothly sucked without clogging the branch conduit 21.

The present invention is not limited to the foregoing embodiment, and can be changed as appropriate within the gist or idea of the invention that can be read from claims, the entire specification, and drawings.

What is claimed is:

1. A suction system comprising:
a branch conduit comprising a main conduit and a suction pipe sleeve branching from the main conduit;
a suction path provided in the suction pipe sleeve, the suction path comprising a first opening communicating with the main conduit;
a working channel comprising an insertion path communicating with the main conduit; and
a liquid feed tube inserted through the main conduit and the working channel, wherein
a first minimum length A, a maximum length B, and a second minimum length C have a dimensional relation of C−B<(A−B)/2, the first minimum length A passing through a first center on a plane of the first opening, the maximum length B passing through a second center on a cross section orthogonal to a longitudinal direction of the liquid feed tube, the second minimum length C passing through a third center on a cross section orthogonal to a longitudinal direction of the insertion path.

2. The suction system according to claim 1, wherein
the main conduit is provided in a longitudinal direction of the branch conduit.

3. The suction system according to claim 1, wherein the suction path has a square cross section, a length of a one side direction of the first opening being the first minimum length A.

4. The suction system according to claim 1, wherein
the suction path has a rectangular cross section, and
a length in a short-side direction of the first opening is the first minimum length A.

5. The suction system according to claim 1, wherein a region of the main conduit near the first opening on a proximal end side with respect to the first opening is provided with a position restriction member, the position restriction member being configured to restrict a position of the liquid feed tube such that the liquid feed tube is positioned away from the first opening by a predetermined distance.

6. The suction system according to claim 5, wherein the predetermined distance is greater than a difference between the second minimum length C and the maximum length B.

7. The suction system according to claim 6, wherein the position restriction member is a rib protruding in a central direction of a cross section orthogonal to a longitudinal direction of the main conduit.

8. The suction system according to claim 6, wherein the position restriction member is a rail protruding in a central direction of a cross section orthogonal to a longitudinal direction of the main conduit, the rail being configured to guide insertion of the liquid feed tube.

9. The suction system according to claim 6, wherein the position restriction member is a ring member including a hole portion that allows insertion of the liquid feed tube.

10. The suction system according to claim 6, wherein the position restriction member is a magnetic body provided on the liquid feed tube, the magnetic body being configured to be drawn by a magnet from outside of the branch conduit.

11. An endoscope comprising:
an insertion portion inserted into a body cavity;
an operation portion provided continuously with the insertion portion;
a branch conduit provided on the operation portion, the branch conduit comprising a main conduit provided in a longitudinal direction of the branch conduit, and a suction pipe sleeve branching from the main conduit;
a suction path provided in the suction pipe sleeve, the suction path comprising a first opening communicating with the main conduit;
a working channel provided in the insertion portion and the operation portion, the working channel comprising an insertion path communicating with the main conduit; and
a liquid feed tube inserted through the main conduit and the working channel, wherein
a first minimum length A, a maximum length B, and a second minimum length C have a dimensional relation of C−B<(A−B)/2, the first minimum length A passing through a first center on a plane of the first opening, the maximum length B passing through a second center on a cross section orthogonal to a longitudinal direction of the liquid feed tube, the second minimum length C passing through a third center on a cross section orthogonal to a longitudinal direction of the insertion path.

12. The endoscope according to claim 11, further comprising:
a distal end portion provided at a distal end of the insertion portion; and
a conduit provided in the distal end portion, the conduit comprising a second opening communicating with the working channel and a third opening provided in a distal end face of the distal end portion, wherein
an inner peripheral face of the conduit is formed to be tapered toward the third opening from the second opening, and an inside diameter of the third opening is equal to the second minimum length C.
